(19) United States
(12) Patent Application Publication
(10) Pub. No.: US 2012/0184027 A1
(43) Pub. Date: Jul. 19, 2012

(54) POLYMER COMPOSITION FOR PHOTOBIOREACTORS

(75) Inventors: Stephan Schuessler, Caldern (DE); Inno Gaul, Rheinbach (DE); Harald Kuppelmaier, Neulussheim (DE); Gerrit Proper, RW Epe (NL)

(73) Assignee: GEORG FISCHER DEKA GMBH, Dautphetal-Mornshausen (DE)

(21) Appl. No.: 13/387,262

(22) PCT Filed: Jul. 1, 2010
(86) PCT No.: PCT/EP10/59344

§ 371 Date: Apr. 5, 2012

(30) Foreign Application Priority Data
Jul. 27, 2009 (EP) 09 166 463.1

(51) Int. Cl. C12M 3/00; G02B 5/26; G02B 5/20; G02B 5/22; B82Y 20/00; B82Y 30/00

(52) U.S. Cl. 435/292.1; 252/582; 252/587; 252/589; 977/773; 977/811; 977/902

(57) ABSTRACT

A polymer composition having a modified absorption and transmission characteristic particularly suited for photoreactors or photobioreactors made of plastic molded parts and exposed to sunlight or suitable artificial light sources, the polymer selectively comprises the following substances or a combination thereof in addition to the conventional standard additives: an inorganic or organic near infrared absorber for absorbing long-wavelength radiation, an inorganic or organic reflector for reflecting ultraviolet radiation, an inorganic or organic reflector for reflecting visible, near infrared, or infrared radiation, an optical brightener or fluorescent dye for converting the absorbed ultraviolet radiation into visible light or fluorescent light, a photochromic dye for modifying the transmission characteristic of the plastic molded part as a function of light intensity, and an antimicrobial additive for preventing or reducing organic deposits in the photobioreactor. The photobioreactor has a helically designed inner surface for efficiently mixing the reaction medium.

### BACKGROUND OF THE INVENTION

The invention relates to a polymer composition with modified absorption and transmission characteristics, suitable especially for photoreactors or photobioreactors composed of polymer moldings which are exposed to sunlight or suitable artificial light sources.

Photoreactors are reaction vessels for performance of photochemical reactions. The reaction media are solutions or suspensions which enter into reactions under the action of light. Photobioreactors are reaction vessels for performance of photobiological reactions similar to photosynthesis in the world of plants. In these photobioreactors, for example, microalgae are used to produce biofuels, for example biodiesel as a form of renewable energy. The use of photobioreactors in the growing of microalgae is also of growing importance in the production of algae concentrates with other fields of application, for example fish farming, the production of food additives, or as a binder or neutralizer of carbon dioxide from offgases from thermal power plants.

High demands are made on the wall materials of the reaction vessel. The material for the walls must have maximum stability to ultraviolet radiation. UV radiation may be harmful to the reaction medium and therefore has to be either retained or reflected, or converted to radiation suitable for the reaction medium (visible light of wavelength 400 to 700 nm).

The material must have the best possible transparency for the suitable radiation. The near infrared radiation (NIR) present in sunlight is crucially responsible for the heating of the photobioreactor and of the algae suspension. Since the growth of algae proceeds optimally only within a particular moderate temperature range, the reactor temperature has to be controlled. The temperature control concept has a crucial influence on the design of the photobioreactor and the efficiency thereof.

A further aim of an optimal photobioreactor arrangement is that the incident radiation usable for algae growth per unit base area is very substantially made usable for algae growth. Maximization of the photobioreactor area per unit base area is therefore an important aim in the optimization of efficiency of photobioreactors. Intelligent layering of photobioreactors with simultaneously effective distribution of the incident radiation over a maximum reactor area must be the aim.

In addition, the material must have maximum mechanical stability. The transparent wall of the reactor must not be soiled by deposits, which means that deposits on the inside of the reactor must be prevented. Because very large reactors, i.e. very long tubes, are required for the performance of the photobiological reactions, the weight and cost of the reaction vessel also play a major role.

EP 1127612 discloses a solar photoreactor. The reaction vessel consists of a jacketed tube system in which the reaction medium is conveyed within the gap between the two tubes. The reaction medium is exposed externally and internally to the solar radiation energy or a suitable artificial light source. For the reaction vessel, glass or plastic tubes transparent to the insolation are proposed.

Proceeding from this prior art, it is an object of the invention to specify a polymer composition for photoreactors, especially for photobioreactors, which makes it possible to optimally adjust the polymer material for the wall of the reactor to the process conditions of the photosynthesis, has minimum weight and guarantees a maximum lifetime.

### SUMMARY OF THE INVENTION

The foregoing object is achieved by a polymer composition with modified absorption and transmission characteristics, suitable especially for photobioreactors composed of polymer moldings which are exposed to sunlight or suitable artificial light sources, wherein the polymer comprises, in addition to the conventional standard additives, optionally one of or a combination of the following substances: an inorganic or organic near infrared absorber for absorption of long-wave radiation, an inorganic or organic reflector for reflection of ultraviolet radiation, an inorganic or organic reflector for reflection of visible, near infrared or infrared radiation, an optical brightener or fluorescent dye for conversion of the absorbed ultraviolet radiation to visible light or fluorescent light, a photochromic dye for light intensity-dependent modification of the transmission characteristics of the polymer molding and an antimicrobial additive for prevention of or reduction in the level of organic deposits in the photobioreactor.

Preferred developments of the invention are evident from the following disclosure.

It is advantageous that the reaction medium in the photobioreactor is protected from ultraviolet radiation. This is achieved by virtue of the reflector comprising titanium dioxide particles with particle sizes in the sub-micrometer or nanometer range. Nanoscale titanium dioxide particles can be used in appropriate size and, given optimal distribution, selectively and with long-lasting efficacy as UV absorbers. A combination of nanoscale titanium dioxide with sub-microscale titanium dioxide has the result that both optimal reflection of UV radiation and broadband protection from visible and NIR light is achieved with a minimum amount of added material, without the use of conventional UV absorber.

It is also advantageous that the heat management in the reactor can be controlled. This is achieved by virtue of the near infrared absorber preferably comprising an inorganic pigment based on rare earth metals. The NIR absorber may either be arranged in homogeneous distribution over the entire wall thickness of the tube, or only in the outer layer in the case of a coextruded tube.

It is additionally also advantageous that the harmful UV radiation is converted to harmless blue or green light. This is achieved by virtue of the optical brightener preferably comprising compounds based on thiophene-benzoxazole.

It is also advantageous that the optimal light intensity is provided in the photobioreactor. This is achieved by virtue of the photochromic dye preferably comprising spironaphthoxazines or naphthopyrans.

It is also advantageous that deposits resulting from algae growth on the inner surface of the reactor are prevented or reduced. This is achieved by virtue of the antimicrobial additive preferably comprising compounds based on carbamate or silver. This is also achieved by virtue of the tube wall having an inner surface free of dead space. This is additionally also achieved by virtue of the inside of the tube wall being in the form of a static mixer. This static mixer also promotes the homogeneous irradiation of the algae suspension and promotes a homogeneous temperature distribution in the reaction medium.

The wall material is modified by the novel polymer composition such that optimal conditions for the growth of the microalgae and for the efficient production of biomass or biodiesel are offered over the entire service life in the photobioreactor. “Optimal” means here that the correct wavelengths from the radiation spectrum are transmitted in the correct intensity, that the harmful wavelengths are reflected or converted to radiation harmless to algae growth, and that the inside of the wall is protected from deposits. The wall material obtains optimal properties for operation in the photobioreactor, which remain constant over the entire service life of the reactor, which means that the transparency of the wall reactor remains constant and the wall does not become matt.

By virtue of the reflector properties of the novel polymer composition, the thus modified wall of the photobioreactor makes an effective contribution to light dilution. The reflected radiation can be reflected onto adjacent photobioreactor surfaces. The amount of incident light per unit base area is thus distributed onto a larger area of irradiated photoreactor.

### BRIEF DESCRIPTION OF THE DRAWINGS

Working examples of the invention are described with reference to the figures. The figures show:

FIG. 1 a section through an inventive tube for a photobioreactor,

FIG. 2 a further section through a tube for a photobioreactor,

FIG. 3 a summary of the test results for heat management in the photobioreactor comprising an inventive polymer composition compared to a conventional polymer,

FIG. 4 an illustration of the effect of the additive for conversion of UV radiation to visible light as compared with a conventional polymer,

FIG. 5 an illustration of the transmission characteristics as a function of wavelength for conventional polymer material as compared with the inventive polymer composition with a suitable addition of titanium dioxide particles and

FIG. 6 an illustration of the transmission characteristics as a function of wavelength for conventional polymer material as compared with the inventive polymer composition with a suitable addition of photochromic additive.

### DETAILED DESCRIPTION

FIG. 1 shows a section of a PVC tube 1. The PVC tube 1 is produced as a polymer molding by extrusion and has, on the inside, a tube wall 2 with an inner surface 3 in the form of a helical line. This influences the flow of the reaction medium as in a static mixer. The spiral grooves 4 or structuring of the inner surface 3 enables efficient mixing of the reaction medium without any great pressure drop in the tubular reactor, even in the case of relatively low flow rates. The inner surface 3 has no dead spaces, i.e. there are no areas where the flow rate is locally reduced such that deposits precipitate out. The inner surface 3 is still easy enough to clean, and the structure does not cause any scattering or coupling losses for the radiation to the reaction medium.

Instead of transparent PVC, it is possible to use any other polymer material whose absorption and transmission characteristics can be modified for the processes in the photobioreactor. Examples of suitable polymers include, as well as transparent polyvinyl chloride, polycarbonate, polymethyl methacrylate, polyolefin, polystyrene, polyethylene terephthalate, polybutylene terephthalate or combinations, partly or fully fluorinated polymers, for example polyvinylidene fluoride or perfluoroalkoxyalkane, copolymers or alloys thereof.

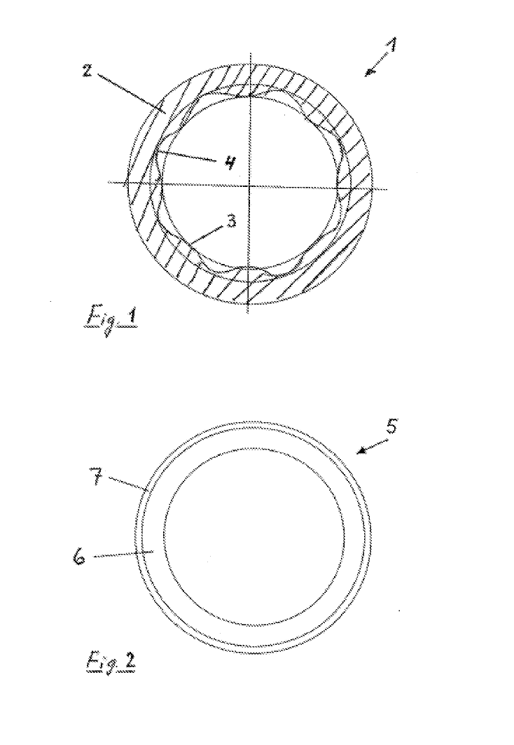

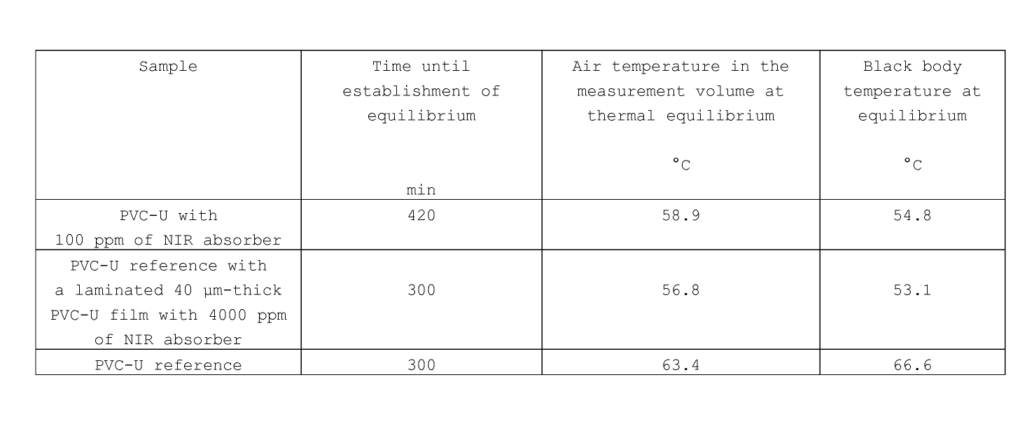

FIG. 2 shows a further section through a tube 5 of a photobioreactor. The tube 5 from FIG. 2 is produced by coextrusion. The tube wall is formed from a relatively thick supporting inner layer 6 and a relatively thin functional outer layer 7. The inner layer 6 may be modified with an antimicrobial additive and with an optical brightener or fluorescent dye. The outer layer 7 is preferably less than 1 mm thick and is additized for modification of the absorption and transmission characteristics. The outer layer 7 comprises the combination of nanoscale titanium dioxide with sub-microscale titanium dioxide and a near-infrared absorber, preferably an inorganic pigment based on rare earth metals.

The movement of the wavelength management into the relatively thin outer layer 7 achieves the following advantages: the main or inner layer 6 is used as a thermal insulator. This reduces the absolute addition of the NIR absorber needed per unit area for the achievement of a particular cooling effect in the outer layer 7. The lifetime of the optical brightener in the inner and/or outer layer 6, 7 is increased significantly, since UV irradiation can be distinctly reduced or controlled. In the inner layer 6, only small contents of conventional UV protection additives are required. The layer structure additionally enables total reflection of the waves filtered out in the outer layer 7. The controlled division of the additives between the inner and outer layers 6, 7 additionally prevents destructive interactions between the different additives, which leads to a longer lifetime of the composite material.

Instead of a coextruded plastic tube, it is also possible to coat or laminate the outside of an existing tube material with the inventive polymer composition, or to laminate it with a thin film. It is also conceivable that existing reactor tubes made of glass or ceramic are covered with such a film.

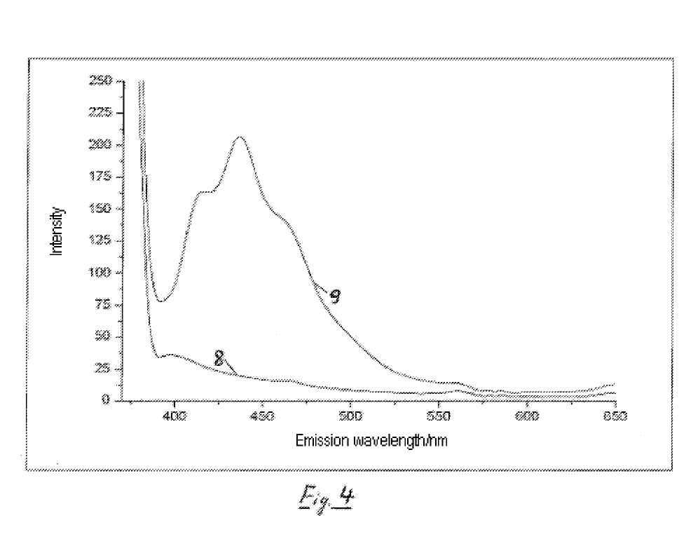

FIG. 3 shows, in a table, a summary of the test results for heat management in the photobioreactor comprising an inventive polymer composition as compared with a conventional polymer. The specimens compared with one another were, in addition to an untreated transparent PVC-U sheet with a thickness of 3 mm, such a sheet containing 100 ppm of an NIR absorber and a composite composed of an untreated sheet with a laminated 40 μm-thick PVC-U film with 4000 ppm of the same NIR absorber. Below the sheets, the time until establishment of equilibrium, the air temperature and the black body temperature in the equilibrium state were measured, in each case in a volume of air at rest. The black body temperature can be regarded as a measure for a reduced heat flow to the medium transported within the tube, and thus demonstrates the efficiency of the NIR absorber.

The test data show that, even in the case of a wall pigmented homogeneously with 100 ppm of NIR absorber, a distinct reduction in temperature is achieved. If, however, the NIR absorber is added in a controlled manner in the relatively thin outer layer, it is possible to distinctly reduce not only the consumption of NIR absorber overall, but also to achieve a further reduction in temperature. In a composite, the inner layer is used as a thermal insulator. The NIR barrier is moved to the outer layer. The NIR absorber added is, for example, Lumogen from BASF.

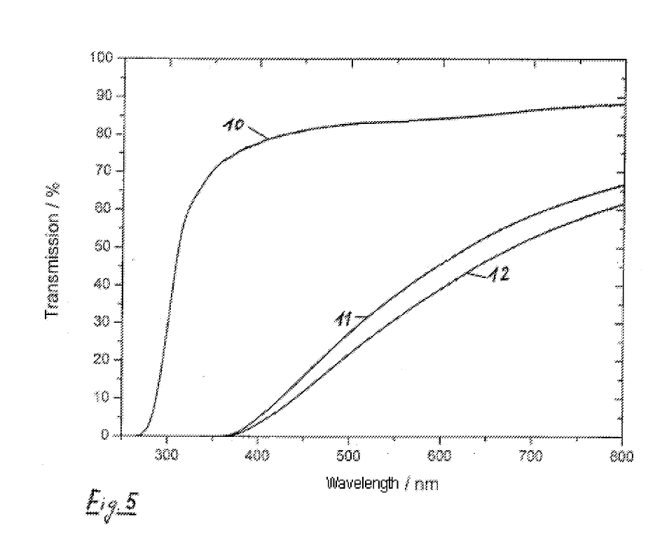

In FIG. 4, the intensity is shown as a function of the wavelength of a reference specimen (curve 8) and a of a sample (curve 9) comprising an optical brightener. The effect of the additive for conversion of UV radiation to visible light is shown here, as compared with a conventional polymer. As the specimen, 0.3 mm-thick PVC-U sheets were pressed. In a specimen, 100 ppm of a UV-active fluorescent dye were added. The emission spectrum of both sheets was recorded after excitation with laser radiation in the UV range.

It becomes clear from the comparison that the fluorescent radiation coincides exactly with the light wavelength range from 400 to 700 nm which is relevant for algae growth. The fluorescent dye added is, for example, Uvitex OB from CIBA.

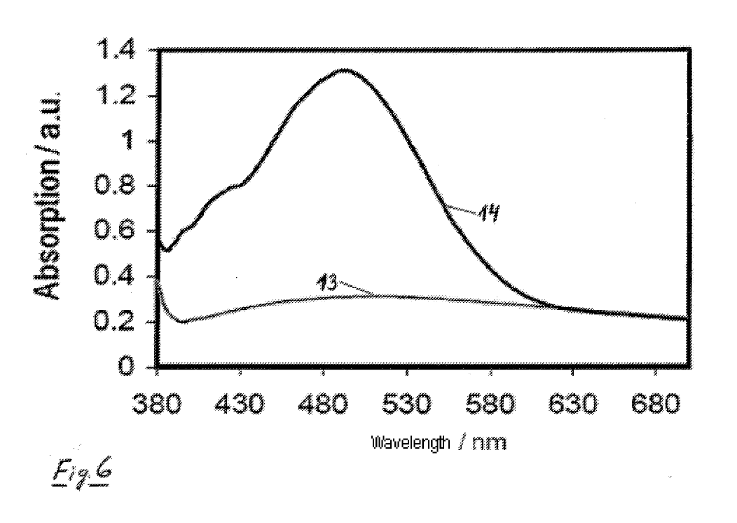

FIG. 5 shows the transmission characteristics as a function of the wavelength for conventional polymer material (curve 10) as compared with the inventive polymer composition with a suitable addition of titanium dioxide particles. As the specimen, 0.3 mm-thick PVC-U sheets were again produced. In one specimen (curve 11), 0.5% by weight of nanoscale titanium dioxide was added. In a further specimen (curve 12), 0.5% by weight of nanoscale titanium dioxide and 0.003% by weight of sub-microscale titanium dioxide were added.

It becomes clear from the comparison that the addition of the nanoscale titanium dioxide alone achieves very efficient UV protection without use of the conventional UV absorber. If a very small amount of sub-microscale titanium dioxide is additionally added, broadband reflector protection for visible and NIR wavelengths is achieved.

FIG. 6 shows the transmission characteristics as a function of wavelength for conventional polymer material (curve 13) as compared with the inventive polymer composition (curve 14) comprising a suitable addition of photochromic dye particles. As the specimen, 0.3 mm-thick transparent PVC-U sheets were again produced. In one specimen (curve 14), 300 ppm of photochromic dye were added and irradiation was effected with a halogen lamp for five minutes. The photochromic dye used is, for example, Reversacol from James Robinson.

It is clear from the spectrum that the action of UV radiation converts the photochromic dye to its color form and it absorbs exactly in the region which is also of relevance for algae growth. This process is reversible. It is thus also possible to achieve light intensity-dependent control of the transmission characteristics of the polymer composition.

Four-week storage tests of specimens of transparent PVC-U tubes of dimensions 63×3.0 mm based on a conventional UV-stabilized formulation comprising 0.1% carbamate-based biocide additive (Fungitrol from ISP) in an algae solution under standard operating conditions (T=25° C., p<1 bar) found a distinct reduction in algae growth compared to a non-biocide-additized pipe sample. The effect of the reduction in growth was estimated at approx. 50%.

The use described here of the polymer composition in the photobioreactor can also be employed in other photoreactors. The tubes are preferably connected by what are called triclamp connectors. Triclamp connections are light, space-saving, and nevertheless easily and rapidly releasable. For this purpose, the pipe end is adhesive-bonded or welded to an angled flank with a collar bush. This type of connection is time-saving and flexible in terms of maintenance. Instead of tubes, it is also possible to produce plates or other polymer moldings comprising the novel polymer composition.

1-15. (canceled)

16. A polymer composition with modified absorption and transmission characteristics, suitable especially for photobioreactors composed of polymer moldings which are exposed to sunlight or suitable artificial light sources, characterized in that the polymer composition comprises a substance selected from the group consisting of:
(1) an inorganic or organic near infrared absorber for absorption of long-wave radiation,
(2) an inorganic or organic reflector for reflection of ultraviolet radiation,
(3) an inorganic or organic reflector for reflection of visible, near infrared or infrared radiation,
(4) an optical brightener or fluorescent dye for conversion of the absorbed ultraviolet radiation to visible light or fluorescent light,
(5) a photochromic dye for light intensity-dependent modification of the transmission characteristics of the polymer molding,
(6) an antimicrobial additive for prevention of or reduction in the level of organic deposits in the photobioreactor, and mixtures thereof.

17. The polymer composition as claimed in claim 16, wherein the reflector comprises titanium dioxide particles with particle sizes in the sub-micrometer or nanometer range.

18. The polymer composition as claimed in claim 16, wherein the near infrared absorber comprises an inorganic pigment based on rare earth metals.

19. The polymer composition as claimed in claim 16, wherein the optical brightener comprises compounds based on thiophene-benzoxazole.

20. The polymer composition as claimed in claim 16, wherein the photochromic dye comprises spironaphthoxazines or naphthopyrans.

21. The polymer composition as claimed in claim 16, wherein the antimicrobial additive comprises compounds based on carbamate or silver.

22. The polymer composition as claimed in claim 16, wherein the polymer is in amorphous or semicrystalline form.

23. The polymer composition as claimed in claim 16, wherein the polymer is selected from the group consisting of transparent polyvinyl chloride, polycarbonate, polymethyl methacrylate, polyolefin, polystyrene, polyethylene terephthalate, polybutylene terephthalate, and mixtures thereof, partly or fully fluorinated polymers, for example polyvinylidene fluoride or perfluoroalkoxyalkane, copolymers and alloys thereof.

24. A photobioreactor comprises a wall formed of a polymer molding wherein the composition of the polymer molding comprises a substance selected from the group consisting of:
(1) an inorganic or organic near infrared absorber for absorption of long-wave radiation,
(2) an inorganic or organic reflector for reflection of ultraviolet radiation,
(3) an inorganic or organic reflector for reflection of visible, near infrared or infrared radiation,
(4) an optical brightener or fluorescent dye for conversion of the absorbed ultraviolet radiation to visible light or fluorescent light,
(5) a photochromic dye for light intensity-dependent modification of the transmission characteristics of the polymer molding,
(6) an antimicrobial additive for prevention of or reduction in the level of organic deposits in the photobioreactor, and mixtures thereof.

25. A photobioreactor as claimed in claim 24, wherein the polymer molding comprises different layers, wherein the layers have different concentrations of substances.

26. A photobioreactor as claimed in claim 24, wherein the polymer molding is in tubular form and has a tube wall with an inner surface in the form of a helical line.

27. A photobioreactor as claimed in claim 26, wherein the tube wall has an inner surface free of dead space.

28. A photobioreactor as claimed in claim 27, wherein an inside of the tube wall is in the form of a static mixer.

29. A photobioreactor as claimed in claim 26, wherein the tubular polymer moldings are in a form such that they can be connected with a triclamp connection.

30. A photobioreactor as claimed in claim 28, wherein the photobioreactor is formed from mineral glass or ceramic.
